(19) United States
(12) Patent Application Publication
(10) Pub. No.: US 2026/0242742 A1
(43) Pub. Date: Aug. 20, 2026

(54) LUNG ORGANOID FOR HIGH THROUGHPUT DRUG SCREENING

(71) Applicant: THE ROYAL INSTITUTION FOR THE ADVANCEMENT OF LEARNING/MCGILL UNIVERSITY, Montréal (CA)

(72) Inventors: Guangyu BAO, Boston, MA (US); Luc MONGEAU, Mont Royal (CA); Alicia Reyes VALENZUELA, Montréal (CA); Mark TURNER, Vancouver (CA); Silvia Marina VIDAL, Montréal (CA)

(21) Appl. No.: 19/162,504

(22) PCT Filed: May 3, 2024
(86) PCT No.: PCT/CA2024/050597

§ 371 Date: Sep. 5, 2025

Related U.S. Application Data
(60) Provisional application No. 63/500,144, filed on May 4, 2023.

(51) Int. Cl. C12N 5/071; C12M 3/06

(52) U.S. Cl. CPC C12N 5/0688; C12M 23/16; C12N 2502/1157; C12N 2502/27; C12N 2513/00; C12N 2533/54; C12N 2533/74

(57) ABSTRACT

It is provided a protocol to induce the formation of lower respiratory tract organoids in only 21 days, the organoids containing markers of both proximal and distal regions. The organoids is obtained by leveraging cellular interactions between airway bronchial epithelial cells and fibroblasts. It is also provided the use of the organoids to study respiratory viral infections with strong similarity to adult lung tissue.

### CROSS-REFERENCE TO RELATED APPLICATION

The present application is claiming priority from U.S. Provisional Application No. 63/500,144 filed May 4, 2023, the content of which is hereby incorporated by reference in its entirety.

### STATEMENT REGARDING FEDERALLY SPONSORED RESEARCH OR DEVELOPMENT

This invention was made with government support under R01 DC005788 and DC018577 awarded by the National Institutes of Health. The government has certain rights to this invention.

### TECHNICAL FIELD

It is provided a protocol to induce the formation of lower respiratory tract organoids from culturing fibroblast cells and airway epithelial cells.

### BACKGROUND

The lung is one of the most complex organs. It is composed of branched tubes with over 58 cell types lining the proximal and distal region. Within the proximal region, epithelial cell sub-types such as goblet, basal and ciliated cells with specialized functions contribute to innate airway defense. One component of innate immunity is mucociliary clearance, mucins secreted by goblet cells trap inhaled pathogens and are removed through the coordinated beating of cilia on ciliated cells, thus preventing pathogens reaching the distal regions of the lungs. In addition, airway epithelial cells can produce and release cytokines directly in response to pathogens, enabling the recruitment of immune cells and the initiation of an immune response.

Basal cells constitute a pool of adult stems cells. They are responsible for the regeneration of the lung after injury. The distal region is formed by alveolar type I (AT1) and type II cells (AT2). The gas exchange between air and blood is performed by AT1 cells and facilitated by AT2 cells through the production of surfactant proteins. Acting as stem cells within the alveolar region, AT2 cells can self-renew and generate AT1 cells throughout the alveogenesis process. Fibroblasts are found abundantly within the proximal and distal region of the lungs. They support the maintenance and survival of AT2 and basal cells through the secretion of Fibroblast Growth Factor. They synthetize extracellular matrix (ECM) proteins, which are vital to efficient gas exchange and support multiple cellular functions. They regulate lung morphogenesis during fetal development. In addition, fibroblasts perform immune functions, such as antigen presentation and bioactive molecules secretion.

To prevent risk of damage during breathing, the lung immune system needs to be gentler than in rest of the body. When particles up to 1 μm of size are carried into the respiratory airways, they encounter alveolar soluble fluids including surfactant and surfactant associated proteins, complement system and alveolar macrophages. The complement system is one of the key players of the immune system. It is formed by a wide variety of plasma proteins which react with each other to opsonize pathogens, induce inflammatory responses that help to fight infection, and directly kill pathogens. Alveolar macrophages are the most abundant phagocytic cell in the lungs. They reduce other immune cells and tone down any inflammation while patrolling the lung surface.

The use of animal models for viral infections may be misleading due to inter-species biological and individual differences, as well as interference with other microorganisms. Conventional 2D monocultures do not accurately mimic tissue organization in vivo, nor cell integrity due to 2D culture adaptation. Typically viral-host interactions involve more than one cell type in the targeted tissue. For instance, severe acute respiratory syndrome-2, SARS-CoV-2 first infects airway epithelial cells, along with ciliated and goblet cells of the proximal lower respiratory tract (LRT). It later infects AT2 cells in the distal LRT as the primary target. Influenza A infects airway epithelium cells lining the LRT, including AT2, thereby destroying key gas-exchange mediators and allowing viral exposure to endothelial cells. Gut, brain, vascular, kidney, and lung organoids have been used to model SARS-CoV-2 and other infectious diseases. Altogether, these organoids have yielded predictive results for drug testing before clinical trials. Their use has been adopted by pharmaceutical companies. But the use of organoids to model the LRT has remained challenging due to the complexity of the lungs. Many lung organoids (LO) used so far to test viral infections are simple. They often consist of only one type of cell representing either proximal or distal regions of the lung, as alveospheres, broncheospheres, tracheospheres or human airways organoids. They are generated by primary cells derived from human or mice embedded in Matrigel and they have a spherical morphology.

More complex lung organoids have recapitulated the entire LRT; but since they are derived from stem cells, they display immature phenotypes and thus cannot be infected with adult viral diseases, including influenza type A and SARS-CoV-2. In addition, these organoids require around 85-150 days to grow, demanding costly and complex culture conditions, as well as being too slow for disease progression.

As the lung is the main target of highly lethal respiratory viruses such as influenza A, and coronaviruses, the creation of physiological relevant in vitro model has been challenging and is desired. No previous model has replicated the sophisticated branching morphogenesis, and multiple-cellular population of proximal and distal regions of the lungs. Ideally, models must be fabricated rapidly to enable studies of emerging viral threats.

There is thus still a need to be provided with new lung models.

### SUMMARY

It is provided a process of producing a lung organoid comprising the steps of culturing lung cells in an hydrogel and a cell culture medium providing a three-dimensional cell growth medium; incorporating airway bronchial epithelial cells into the hydrogel at a ratio of at least 2:1 of epithelial cells:lung cells; and co-culturing the airway bronchial epithelial cells with the lung cells in the hydrogel forming the organoid.

In an embodiment, the lung cells are fibroblast cells.

In an embodiment, the fibroblast cells are lung fibroblast cells.

In another embodiment, the airway bronchial epithelial cells are primary cells or cell lines derived cells.

In a further embodiment, the primary cells are patient-derived primary human bronchial airway epithelial cells (pHBE).

In an embodiment, the primary cells are from a patient with asthma.

In an embodiment, extracellular matrix (ECM) proteins are incorporated into the hydrogel prior to incorporating the airway epithelial cells.

In another embodiment, the ECM proteins are laminin, collagen IV, entactin, heparin sulfate proteoglycans, or a mixture thereof.

In an embodiment, the cell culture medium comprises a mixture of Minimal Essential Medium (MEM) and Dulbecco's Modified Eagle Medium (DMEM) for organoids derived from immortalized cells (LOi), and Bronchial Epithelial Cell Growth Medium (BEGM) in combination with DMEM for organoids derived from primary cells (LOp)

In another embodiment, the mixture of MEM:DMEM or BEGM:DMEM is at a 1:1 ratio.

In another embodiment, the hydrogel comprises alginate, gelatin or a mixture thereof. Preferably, the hydrogel comprises 2% alginate and 5% gelatin.

In an embodiment, the co-culturing of the airway epithelial cells with the lung cells forms spheroids after three days.

In another embodiment, the co-culturing of the airway epithelial cells with the fibroblast cells produces matured organoids in 21 days.

In another embodiment, the process described herein further comprises incorporating a non-epithelial cell type in the organoid.

In an embodiment, the non-epithelial cell type is monocytes or macrophages.

In another embodiment, the lung organoid is produced using bioprinting.

As encompassed herein, it is provided an isolated lung organoid produced by the process described herein.

In an embodiment, the isolated lung organoid comprises an heterogeneous cellular organization.

It is also provided a kit comprising a surface, an hydrogel coated on said surface, and the organoid described herein grown in the hydrogel.

In an embodiment, the kit can comprise at least one microfluidic channel casted in the hydrogel. Particularly, the kit comprises at least one microchannel lined with lung endothelial cells forming a tube and at least one microchannel lined with airway epithelium cells. In an embodiment, the at least one microchannel lined with airway epithelium cells is embedded with extracellular matrix (ECM) proteins and fibroblasts to form the organoid. In a further embodiment, the at least one microchannel lined with airway epithelium cells is perfused with air to form an air-liquid interface.

It is further provided the use of the organoid and/or the kit encompassed herein to characterize a respiratory viral infection and/or measure therapeutic drug activity against the respiratory viral infection.

In an embodiment, the viral infection is from influenza A, COVID-19, Nipah virus, or Ebola virus to measure therapeutic drug activity against the respiratory viral infection. Alternatively, the viral infection is from influenza H1N1, influenza H3N2 and SARS-CoV-2.

### BRIEF DESCRIPTION OF THE DRAWINGS

Reference will now be made to the accompanying drawings.

FIG. 1 illustrates establishment of human lung organoids using cell lines. A) Lung tissue morphogenesis. B) Protocol for organoid formation. C) Maturation of Lung Organoids (LO) over time. On top: brightfield pictures. Magnification 4×. Scale Bar 500 μm, Bottom. Immunofluorescence pictures of LO. The size of the organoid increases over time. Magnification 10×. Scale Bar 500 μm. D) Effect of excessive fibroblast density in organoid formation. LO show lack of interconnection, and loose packing. Magnification 10×. Scale Bar 50 μm. E) Reproducibility of the method using three independent cultures. Although there are some variations in the morphology, the pattern between cultures remains the same. At day 3, spheroids of epithelial cells are formed, at day 16 tube formation occur, and at day 21, the organoid increase in thickness and contain multiple branches. Magnification 10×. Scale Bar 500 μm. Red-F-actin. Blue-Dapi. F) length of the organoid over time. The organoid reached a macro-scale size of around 1200 μm at day 21.

FIG. 2 illustrates lung organoids growth with 3dGRO medium at day 16. Micrographs 10×. Scale Bar 500 μm.

FIG. 3 illustrates lung organoids growth with human Vocal Fold Fibroblasts (hVFF) at day 16. Brightfield micrographs 4× (left panel). Scale Bar 500 μm. Immunofluorescence micrographs. Magnification 10×. Scale Bar 100 μm.

FIG. 4 illustrates microphotographs of LOi at day 21 using FIB microscopy showing in (A) magnification 500× or (B) scale Bar 598×. Scale Bar 50 μm. Arrows indicate the airway epithelial cells in a tube-like structure. Also seen is heterogenous structures of stratified squamous epithelium (multiple layers) and pseudostratified squamous epithelium (single cell layer).

FIG. 5 illustrates microphotographs of LOi at day 21 using FIB microscopy with magnifications of 5000×. Scale Bar 5 μm. Arrows indicate microvilli structures.

FIG. 6 illustrates differentiation of airway epithelium into multiple sub-types of epithelial cells in lower respiratory tract lung organoid. A) Immunofluorescence of LO expressing AT2 markers. Right: Magnification 10×, Left and B Magnification 20×. Scale Bar 500 μm. SFTPC-Red, SFTPB-Green, Nuclei-Blue. B) Micrographs of LRT-LO expressing PDPN, HOPX, MUC5AC, and MUC5B. Magnification 10×. Scale Bar 500 μm. C) qPCR of lung fate genes in LRT-LO organoid and CFBE monolayers, analyzed with Two-way ANOVA p<0.05. D-E) RNA sequencing showing proximal and distal markers in cell monolayers and organoids, wherein p adjust<0.05.

FIG. 7 illustrates generation of human Lower Respiratory Tract Organoid using patient derived cells. A) Development of the LRT-LO over time, from day 4 to day 21, white lines depicted primary branches, while lines depicted secondary branches. Micrographs 4× magnification. Scale Bar 500 μm. B) Organoids derived by female patient cells. Micrographs of different regions of the organoid at day 21. Magnification 10×. Scale Bar 200 μm. C) Proximal and distal markers of LRT-LO. Micrographs 10× magnification. Scale Bar 500 μm. D-E) RNA sequence analysis showing in D) proximal markers, and E) distal markers of multiple T-test comparison, p.adjust<0.05 in comparison to IMR-90*, and in comparison to HBE.

FIG. 8 illustrates a heatmap showing genes associated with respiratory system development pathway: Gene Ontology: 0060541. Differential expressed genes analysis, comparing cell monolayers (HBE, IMR-90) and LOp, (p adj<0.01 & abs (log 2 Fold Change shrink=>4).

FIG. 9 illustrates a heatmap showing genes associated with alveolus development in all samples. Differential expressed genes analysis, comparing cell monolayers (HBE, IMR-90) and LOp, (p adj<0.01 & abs (log 2 Fold Change shrink=>4).

FIG. 10 illustrates a heatmap showing genes associated with tube development pathway: GO: 0035295. Differential expressed genes analysis, comparing cell monolayers (HBE, IMR-90) and LOp, (p adj<0.01 & abs (log 2 Fold change shrink=>4).

FIG. 11 illustrates a comparison of proteomic profile of each type of lung organoids (LOp) and LOi. A) upregulated proteins in LOp with high specificity to the lung showing connections on pathways, and in B) subset of proteins from the immune system upregulated in LOp organoids in comparison to cell lines.

FIG. 12 illustrates a volcano Plot shows the upregulated proteins in LOp (right) and the downregulated proteins (left) in comparison to pHBE monolayers. Generated by VolcaNoseR

FIG. 13 illustrates upSet plots visualizing the proteins shared between adult lung tissues and LOp (A) or LOi (B).

FIG. 14 illustrates a summary of overrepresented terms, categorizing proteins based on their biological roles or properties in LOp and LOi.

FIG. 15 illustrates over expression of host genes for early viral replication and viral receptor proteins on the organoid facilitates influenza H1N1 infection into Human Lower Respiratory Tract Organoids. A-E) RNA sequence data. T-test p. adjust<0.05. A) Genes associated with severity of COVID19, B) Interferon Induced Transmembrane Genes. C-E) Genes on host associated with entry and replication of influenza. C) Genes involved in IP3-PCK pathway, D) Genes associated with COPI vesicles. E) Genes associated with endosomal uptake and maturation. F) Micrographs of LO-immortalized at day 7 expressing TMPRSS-2 and ACE2 for entry and priming of virus infection. Magnification 10×. Scale Bar 100 μm. G) Gene expression of TMPRSS-2 and ACE2 in organoid and CFBE monolayers. n=3, p<0.05. H) Schematic representation of viral infection in the organoids. I) Viral titers by plaque assay using two infection condition (n=2). No significant difference was found. J) Gene expression of viral gene H1N1 in homogenized organoids infected under two different conditions (n=2). Viral replication was found inside and in the organoid supernatant. K) L) Viral copies on organoids infected with SARS-COV-2 under MOI:0.01, and 0.001.

FIG. 16 illustrates representative images of asthmatic organoids achieved by incorporation of IL-10 into the medium. No fibroblasts were incorporated into the culture. Magnification 10×. Scale Bar 500 μm.

FIG. 17 illustrates bioprinting of lung organoids, showing in (A) schematic representing the overall protocol. In (B) AlGe droplet with 2 mm of height. In (C) representative lung ‘organoid’ on day 21. Magnification 2×. In (D) robustness of ‘organoids’ which can be handled with tweezers. In (E) lung ‘organoids’ growth on spinning bioreactor. In (F) Organoids were retrieved after being cultured on a spinning bioreactor for 21 days.

FIG. 18 illustrates a variation the lung organoids provided herein wherein monocytes (THP-1) were cultured on top of 8 μm porous membranes using two different cell densities: 1:1000, and 1:10000, in relationship to airway epithelial cells (CFBE) during 24 h or 72 h after 4 days post organoid formation. Arrows show cell debris from the main structure. Magnification 10×. Scale Bar 500 μm.

FIG. 19 illustrates functions of enriched genes (cluster 1) in both type of organoids LOp and alveolospheres of the prior art and their significance (p adjusted).

FIG. 20 illustrates function of enriched genes (cluster 2) in LOp in comparison to alveolospheres, and its significance (p adjusted).

### DETAILED DESCRIPTION

In accordance with the present description, there is provided a protocol to induce the formation of lower respiratory tract organoids in only 21 days, the organoids containing markers of both proximal and distal regions. The organoids is obtained by leveraging cellular interactions between airway epithelial cells and fibroblasts in a compliant substrate within ECM proteins. It is also provided the use of the organoids to study respiratory viral infections as well as potential therapeutic drugs against them.

Most existing lung organoids represent only one region of the lung, either proximal or distal. They require primary cells from donors which hampers their scalability. The most complex lung organoids require over 85 days to fully grow. Their phenotype remains fetal, which makes them inappropriate for studies of adult viral diseases. Here, a new protocol to induce the formation of lower respiratory tract organoids in only 21 days is described. The organoids contains markers of both proximal and distal regions. The structure is obtained by leveraging cellular interactions between 2 cell types: airway epithelial cells (immortalized and primary) and fetal fibroblasts. Cell differentiation in multiple epithelium sub-types is induced through appropriate control of the microenvironment. The resulting organoids were positive for goblet, ciliated, basal, and alveolar types I and II cell populations. Their morphology mimicked lung tissue morphogenesis over time, forming unprecedented macrostructures over 1 cm. Tandem Mass Tags (TMT) revealed the expression of proteins in the organoids that were not present in the starting cell lines. Some of these proteins are involved in tissue development, cell differentiation, and tube formation. The organoid proteome was found to closely resemble that of adult lung tissue. The upregulation of proteins related to the innate immune system was observed presumably to enhance responsive defense against potential threats. As a proof of concept, the ability of the organoid model to be infected was demonstrated with influenza H1N1, and SARS-COV-2, indicating the potential for its use in the study of viral infections such as Avian Influenza, and other strains of COVID-19, and the Nipah virus. The ability to study these viruses in a physiologically relevant model could lead to a better understanding of their pathogenesis, and to the development of new therapies in vitro.

The organoids encompassed herein replicate the branching morphogenesis of the LRT, express markers of cell types critical in lung function and infection/immune response. The organoids require only 21 days to mature displaying adult phenotype. Such fast-growing complete organoids were obtained from airway epithelium and fetal fibroblasts by mimicking cell signaling and communication.

Proteome profiles of initial cell monolayers, organoids, and adult lung tissue were compared to establish a reliable benchmark between different in vitro lung models. The results indicated that the organoids had a greater similarity with adult lung tissue than cell monolayers, justifying their use in drug discovery and disease modelling. Organoids upregulated proteins with innate immunity function and specificity to the lung and tissue development, indicating the importance of cellular interactions and providing a more physiological response against pathogens. The organoids upregulated genes associated with influenza infection type A, and severity of COVID-19, therefore the model was tested for viral infection with influenza type A, and SARS-COV-2.

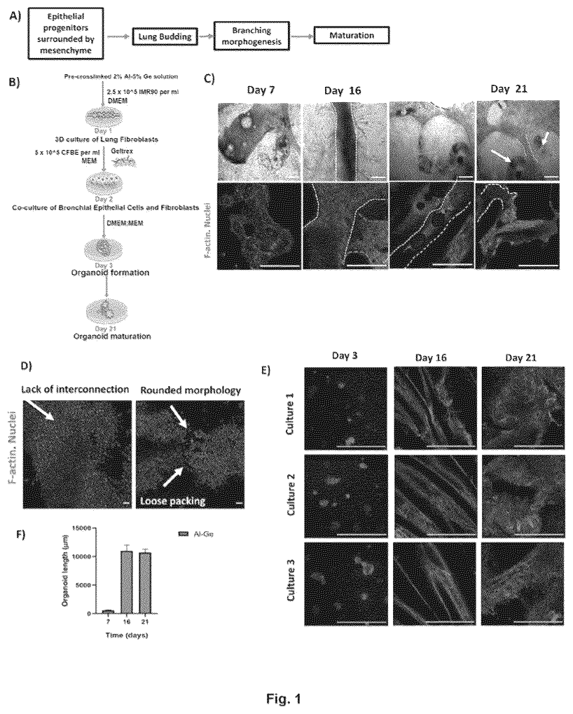

To induce the formation of human lung organoids, airway epithelial cells were co-culture along with fibroblasts and microenvironmental cues, in specific 1) 3D cellular culture in 2) a compliant substrate with stiffness representative of the lung, which was coated with 3) ECM proteins. FIG. 1A shows the morphogenesis process of the lung in vivo. Epithelial progenitors surrounded by mesenchyme, triggers the process of lung development. To mimic this process, the lung fibroblast finite cell line (IMR-90) was encapsulated into hydrogels consisting of 2% Alginate and 5% Gelatin (AlGe). Any hydrogel which favored the growth and proliferation of fibroblasts would lead to disruption of the organoid, is encompassed herein. However, small concentrations of fibers or peptides inside alginate and gelatin such as Arginine-Glycine-Aspartic Acid (RGD) can be used. Generally speaking, neutral and soft hydrogels can be used for the generation of the organoid. Alginate itself is a neutral hydrogel, less stable than its combination with gelatin, but able to be used for encapsulation of fibroblasts.

The following day, ECM proteins such as laminin, collagen IV, entactin, and heparin sulfate proteoglycans were incorporated into the top of the hydrogel. Geltrex was used, but other commercial available products such as Matrigel, or a preparation of decellularized ECM of animals may lead to similar results. Human bronchial epithelial cells, acquired from the immortalized cystic fibrosis bronchial epithelial CFBE cell line (CFBE41o-), were then seeded on top. An overview of the protocol is shown in FIG. 1B. The ratio in which fibroblasts need to be incorporated in proportion to CFBE plays a critical role for the development of the organoid. Fibroblasts initial maximum density should be half than initial density of the epithelial cells, in healthy conditions. When modelling a disease in which airway epithelial cells have a inflammatory condition from the starting such as asthma, cystic fibrosis or emphysema. The initial density of the fibroblasts needs to be further reduced.

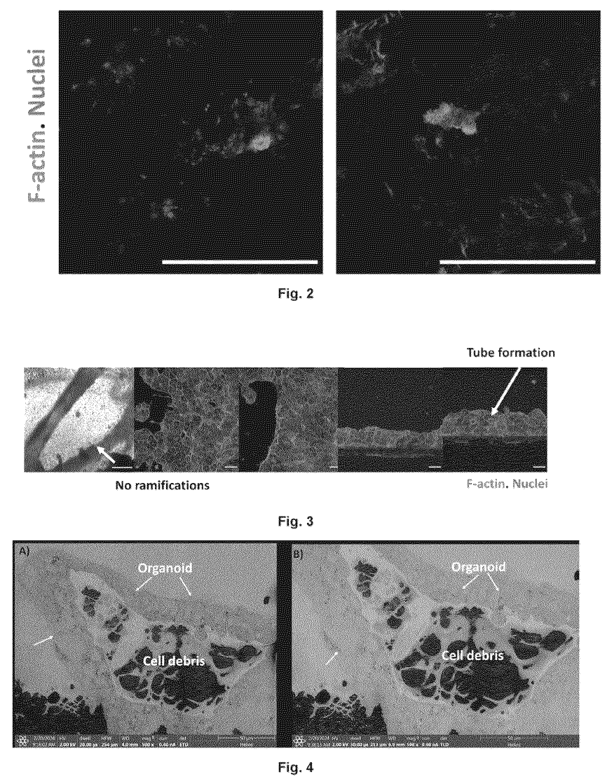

The co-culture gave rise to the formation of spheroids at day 3. At day 7, the epithelium layer elongated to initiate lung budding and, primary bronchial branches were observed (FIG. 1C). At day 16, tube formation and branching morphogenesis occurred (FIG. 1C). By day 21, these branches increased in diameter and length, and multiple ramifications were formed. As the branches further elongated, their thickness decreased. Cell clusters were found positive for surfactants and Podoplanin, typical markers of alveolar cells (FIG. 1C and FIG. 3) located at the extremities of these ramifications. The lower respiratory tract lung organoid (LRT-LO), including distal regions, were thus generated after 21 days of culture. The mesenchymal support of fibroblasts as feeder cells was essential to promote the development of the organoid. The 3D cellular structure can be appreciated during tissue morphogenesis (FIG. 1C). The F-actin of every cell is tightly interconnected, thereby supporting the complex lung morphology. An interdependency was found between the organoid formation and the seeded density of lung fibroblasts. When their number is equal or greater than the number of airway epithelial cells, fibroblasts become the dominant cell type and disrupt organoid formation and F-actin elongation (FIG. 1D). This results in a loosely packed, rounded cell morphology, with few F-actin interconnections (FIG. 1D). Similar outcome was also observed following the use of different substrates such as hyaluronic acid and gelatin methacryloyl (GelMA) which promote the growth of fibroblasts. The formation of the organoid was also investigated by modifying 2% alginate with the tripeptide sequence Arginine-Glycine-Aspartic Acid (RGD) coupled with 5% Gelatin (Al-RGD-Ge). The resulting organoids shown tight ZO-1 and E-cadherin junctions. The viability of the organoids was not affected by this modification relative to that of AlGe alone. In both cases dead cells were found surrounding the structure of the organoid, but not in the organoid itself, suggesting that some fibroblasts were dying during the culture. The stiffness of both substrates was found to be within the range of values previously reported for lung tissue. The protocol was optimized using a combination of Minimal Essential Medium (MEM) and Dulbecco's Modified Eagle Medium (DMEM) in a 1:1 ratio, without supplementation with growth factors or signaling molecules. Using DMEM alone may be detrimental as it favored the growth of fibroblasts, MEM alone favors the growth of airway epithelial, so it may results in similar outcome than our selected media. The organoids cultured in 3dGRO™ organoid cell culture medium were found to have a smaller size and a smaller number of branches and interconnections (FIG. 2) than those cultured in MEM: DMEM. The 3dGRO™ medium has a higher cost than the selected media, due to the incorporation of signaling molecules such as Wnt3a, R-spondin-3 and Noggin. In contrast the protocol provided herewith requires basal medium, since the signaling molecules and growth factors are directly produced by IMR90. In addition, the reproducibility of the method was evaluated, monitoring three different cell cultures until day 21 using immunofluorescence (FIG. 1E). The method was found to be reproducible; in each experiment, tube formation was observed at day 16 (FIG. 1E). The original source of mesenchymal cells is an important condition for the formation of the lung organoid. When vocal fold fibroblasts were used, two tubes were formed without any additional branches. This suggests organoid specific phenotype and fate (FIG. 3). Any other lung fibroblast cell line will produce similar results and is encompassed herein. The organoid was grown on submerged medium. The organoids formed with the protocol encompassed herein show a sophisticated and branched morphology after only 21 days. The present approach guided the macroscale self-organization of the organoids. The length reached by the organoids at day 21 was 10070±60.82 μm (FIG. 1F). This constitutes the first macroscale organoids that were formed in isolation within their microenvironment, and cell signaling generating robust and interconnected structures which follow the lung morphogenesis.

Organoids derived from immortalized cells (LOi) were visualized using Focused Ion Bean Microscopy (FIB) following a 21-day culture period. The organoid was imaged at varying higher magnifications, including 500×, and 598× as depicted in FIG. 4. These close observations revealed intriguing features, including the alignment of cells within the organoid structure and discernible morphological variations among individual cells. Particularly noteworthy were the distinct cellular arrangements observed within different regions of the organoid. Some regions exhibited stratified airway epithelial cells, characterized by multiple layers of epithelial cells stacked upon each other, while in other regions, a pseudostratified arrangement was apparent, where only a single layer of epithelial cells was observed. These findings underscore the complexity and heterogeneity of cellular organization within the organoid, shedding light on its structural diversity. This finding challenges the notion of cellular homogeneity within the organoids and underscores their dynamic nature, potentially reflecting a degree of cellular plasticity and functional diversity. Notably, the identification of microvilli, which are typically associated with brush cells in the lung, further highlights the organoids' ability to mimic essential characteristics of native lung tissue. This aspect emphasizes their relevance as a model system for studying respiratory physiology and pathology. Autophagy is the process of degrading cytoplasmic components within lysosomes. This process, which is abundant in LOi, serves various physiological functions, including adaptation to starvation, clearance of intracellular proteins and organelles, development, anti-aging, elimination of microorganisms, cell death, tumor suppression, and antigen presentation. Interestingly, autophagy has been associated with the regulation of lamellar body formation. Lamellar bodies are concentric structures loaded with surfactant proteins specific to AT2 cells. While the reason for the high presence of autophagy in the organoids is not certain, one possibility could be its involvement in the development of these organelles.

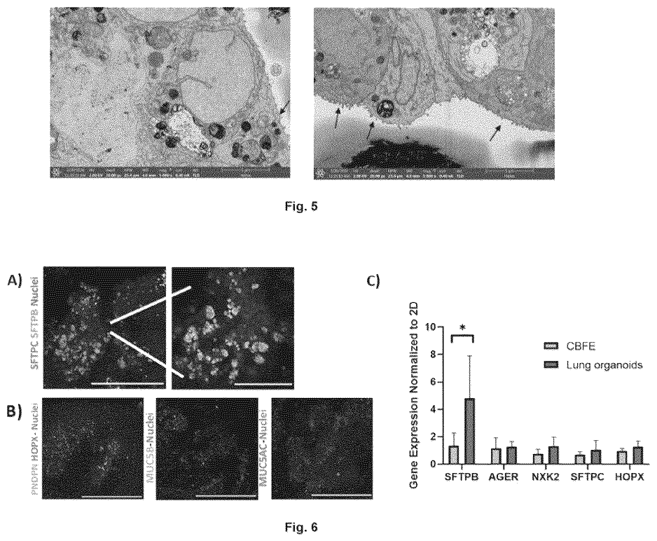

As seen in FIG. 5, detailed subcellular structures are observed, indications of autophagy, and the presence of microvilli structures (indicated with arrows). Microvilli are microscopic membrane cellular protrusions which are involved in the process of absorption of nutrients and cellular secretion. They have a significant function in transmitting signals that trigger immune responses. The emergence of microvilli suggests a level of cellular differentiation and specialization within the organoids, despite their origin from cell lines that are traditionally considered homogeneous. This finding challenges the notion of cellular homogeneity within the organoids and underscores their dynamic nature, potentially reflecting a degree of cellular plasticity and functional diversity. Notably, the identification of microvilli, which are typically associated with brush cells in the lung, further highlights the organoids' ability to mimic essential characteristics of native lung tissue.

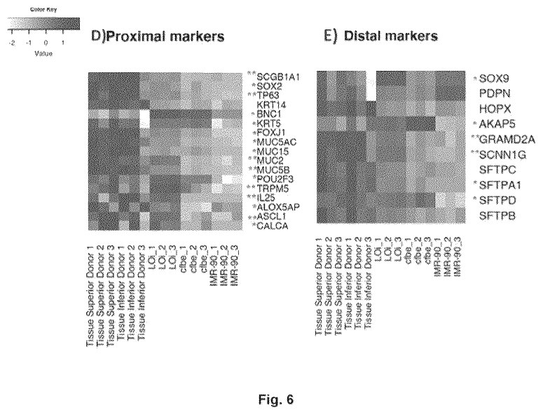

The cellular composition of LRT-s was investigated to gain insights into the differentiation process that led to the formation of the organoids through the cellular interactions between lung fibroblasts and epithelial cells. To achieve this, a combination of immunostaining, qPCR, and RNA sequencing was used to detect markers indicative of the proximal and distal regions of the lung. Distal markers for AT2 and AT1 cells were identified in the organoid structures at day 21. Surfactant Protein-B (SFTPB) and Surfactant Protein-C (SFTBC) were observed at the tip of the ramifications of the organoids by immunofluorescence (FIG. 6A). Homeodomain-only protein (HOPX) was also identified by immunofluorescence, and along with Podoplanin (PDPN) was used as a marker of AT1 cells (FIG. 6B) in the organoid culture. Distal markers were also evaluated and found in Al-RGD-Ge derived organoids.

The expression of lung markers in the organoid was confirmed by qPCR (FIG. 6C). Gene expression of both surfactants was also found in the monoculture of CFBE in ALI interface (FIG. 6E). However, the expression of SFTPB in the organoids was significantly higher. Other distal markers of the lung were identified into the LRT-LO organoid by qPCR, including homeobox protein Nkx-2 (NXK2) and Advanced Glycosylation End-Product Specific Receptor (AGER) (FIG. 6C), confirming the lung fate of the model. Proximal markers were also identified in the organoid without a particular organization. FIG. 6B shows micrographs of the organoids showing mucin 5AC (MUC5AC), and mucin 5B (MUC5B) as markers of goblet cells.

RNA sequence was used to validate the results. FIG. 6D shows the proximal markers found in the organoids and their comparison with cell monolayers. Markers of basal cells (Cytokeratin 14, p63, and BNC1) were absent from the initial cell monolayers (FIG. 6D), as expected given their stage of immortalization. Although the expression of these specific genes increased after the organoid was formed, relative to CFBE and IMR-90, the increment was not significant, except for TP63 which expression was upregulated with respect to CFBE and IMR90. This could perhaps indicate that the microenvironment plays a beneficial role in the genome even after cells are immortalized. Markers of secretory cells (SGB1A1+) from the proximal region, goblet (MUC5B+, MUC2+), brush (TRMP5+, IL25+, ALOX5AP+), and PNECS (ASCL1+), were all undetectable or minimal in cell monolayers, but they were all significatively upregulated in LOi (FIG. 6D). Similarly, secretory cells (SGC3A2+) were upregulated in the organoids These results suggest that, regardless of the immortalization of the cells, CFBE have the potential to change their phenotype according to their culture conditions, albeit with some limitations. Markers for AT2 cells (SFTPC+, SFTPB+, SFTPA1+, SFTPD+) were not significantly upregulated in the organoids with respect to CFBE, but SFTPA1+ and SFTPD+ increased in LOi in respect to IMR-90. Interestingly, the expression of SFTPC, and SFTPB in LOi was confirmed by parallel immunofluorescence. Based on these findings, it is inappropriate to attribute the expression of AT2 markers in the CFBE cell line to differentiation into AT2 cells. Instead, it is more likely that the immortalization process induces changes in the cellular characteristics, leading to the expression of these markers. Based on the data presented, it is apparent that LOi exhibits a distinct lung tissue phenotype. When compared to the input cell types, LOi primarily regulates markers associated with the proximal region of the lung. One notable difference between LOi and LOp is the lack of upregulation of AT2 and AT1 markers in LOi. The upregulation of markers of brush cells is consistent with the microvilli structure identified with FIB microscopy (FIG. 5) This further support the idea that there is some degree of plasticity on these organoids regardless of their cell origin.

Arguably, PDNP, and HOPX are the best markers to evaluate AT1 differentiation. However, some studies have shown than these genes are expressed by other cell types in the lung. By RNA sequence, both HOPX and PDPN are expressed on IMR-90 and CFBE monolayers, as well as in the organoids. In fact, PDPN was higher expressed on IMR-90 monolayers (FIG. 6E). Further, two additional markers, Gram domain-containing 2A (GRAMD-2A), and Sodium Chanel Epithelial 1 Subunit (SCCN1G), were found in the organoid.

It appears that most of the cells into LRT-LO have formed new hybrid structure organoids generating a model that co-expresses multiple markers of the lung, that closely mimic in vivo cell communication and cell signaling. Flow cytometer was used to confirm that cells within the organoid were positive for more than one cell marker. Airway epithelial cells used for the organoids appears to undergo to a reversible transition process to their mesenchymal stage. This process is essential for the development of complex three-dimensional organ structures. It has been previously reported this process is instrumental for cells to attain a hybrid phenotype.

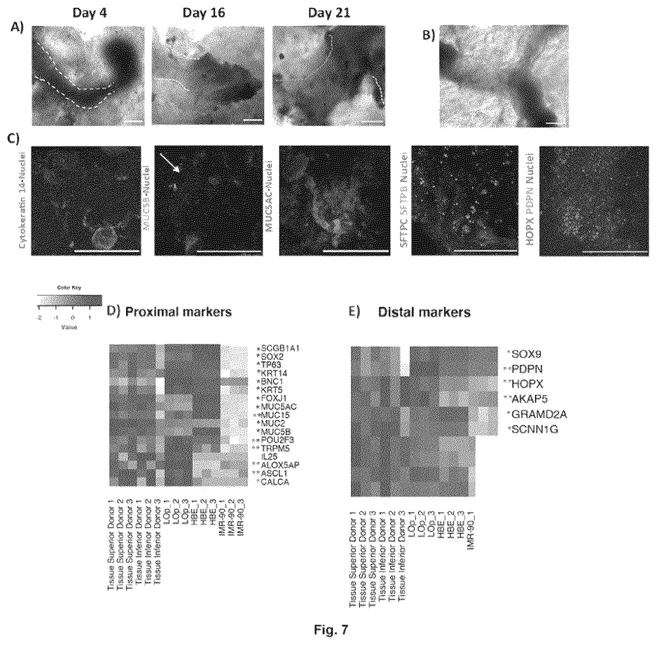

The organoid model generated using immortalized Cystic Fibrosis Bronchial Epithelial Cells (CFBE) had shown positive results in replicating the morphogenesis of lung structure and in the differentiation of multiple airway epithelial cells. As provided herewith, the provided protocol was replicated with patient-derived primary human bronchial airway epithelial cells (pHBE) to explore whether the physiological relevancy could be further improved. The use of cells directly from patients is a key step for its use in personalized medicine, theoretically allowing to assess the individual's reaction to diseases and drugs in vitro. Cells isolated from human lungs of both sexes (a 53-year-old female and a 51-year-old male) were used. The development of the organoids were monitored with brightfield microscopy. FIG. 7A shows the growth of organoids derived from male pHBE cells over time. Tube formation was observed from day 4. It increased in length and dimeter by day 16 and day 21. Fewer ramifications were observed with these types of organoids than with the ones derived from immortalized cells. FIG. 7B shows a representative micrograph of organoids generated by female-derived pHBE cells. Overall female cells generated more robust organoids, and with more ramifications, but this difference is possibly due to the variability between donors and not sex. Next, proximal and distal cell markers were identified using immunofluorescence. FIG. 7C shows positive cells for proximal markers C14 for basal cells, MUC5AC, and MUC5B for goblet cells, and distal markers such as SFTPB, SFTPC for AT2 cells, and PDPN and HOPX for AT1 cells. RNA sequence helped to validate these results. Goblet cell markers were found more abundantly in pHBE ALI culture, than in the organoids. Basal cells were found in these organoids, C14 gene expression was significantly more abundant in the organoids than in the pHBE, but p63 was found in very similar levels in both pHBE and LO. Neither basal cell nor goblet cell markers were found in IMR-90, as expected. SOX2, used as a marker of multipotency and proximal region of the lung was found in both pHBE, and LO, interesting this multipotency marker decreases in organoids formed with primary cells suggesting further differentiation. FOXJ1 was also found in the organoids in lower levels in comparison to pHBE. Primary human bronchial epithelial cells cultured in ALI have been used extensively as a model of proximal region of the lung. The initial cell monolayer contains epithelial sub-population that with ALI and time fully differentiate into goblet and ciliated cells. Based on the RNA sequence data, it can be assumed that pHBE is a superior model for proximal region of the lung, however lung organoids express markers of both regions, and as such it is expected a decrease in the abundance of proximal markers. Nevertheless, lung organoids also express Secretoglobin family 3A member 2 (SCGBA3A2) indicating secretory cells, which aren't present on pHBE or IMR-90.

FIG. 7E shows distal markers of the lung and their gene expression in comparison to cell monolayers. All markers assigned for AT1 cells, PDPN, HOPX, GRAMD-2A, and SCC1NG were presented in the organoid. HOPX, and PDPN were higher expressed in the organoid than in pHBE cell monolayer. In addition, all surfactant proteins were expressed in the organoid, and except for SFTPB, they were more abundant than in pHBE. Similarly, SOX9, indicating distal phenotype, was highly expressed in the LO, while in pHBE and IMR-90 the gene expression was significantly lower. ETS variant transcription factor 5, was also found in the organoids.

Finally, flow cytometer confirmed the presence of goblet cells, basal cells, and AT2. All data together indicates proximal and distal cell differentiation within the organoids. Markers of goblet, ciliated, basal, and secretory cells were found in the organoids, as well as distal markers such as surfactant proteins for AT2, and PDPN, HOPX, and GRAMD-2A for AT1 cells. This is the first time that human lung organoids derived by adult bronchial airway epithelial cells mature to further differentiate into distal regions of the LRT and are capable to generate secretory cells. This data confirms the application of the provided protocol to form multiple cell populations of lung organoids using primary cells. Using this cell type, improved the cell differentiation, possibly due to the pool of basal cells at the starting of culture. There is previous evidence supporting than human alveolar cells can transdifferentiate into basal cells due to fibroblastic signaling. Accordingly, in these organoids basal cells are the ones that further transdifferentiate into AT1 and AT2 cells. Organoids generated by primary cells may be useful for personalized medicine and contribute to the future establishment of biobanks lung organoids.

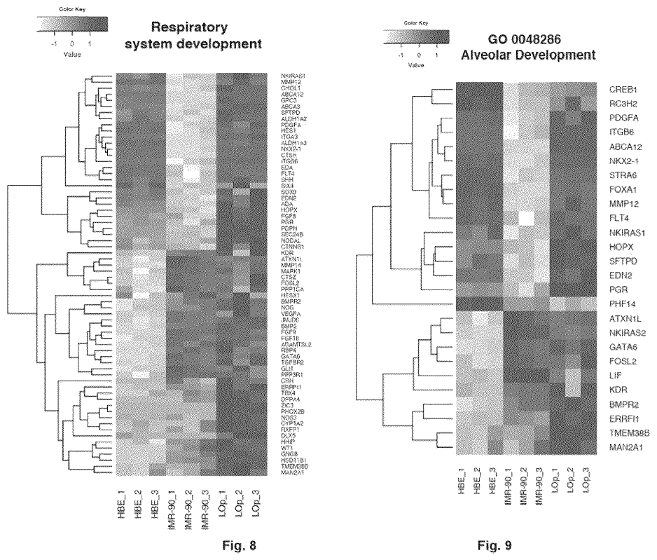

FIGS. 8-10 show several Biological Process ‘terms’ (set of genes associated with specific functions) identified from the functional enrichment analysis associated with respiratory system development, tube development and lung alveolus development. Many genes from each term were upregulated in the organoid versus cell monolayers.

As shown in FIG. 8, many genes associated with respiratory system development appear in HBE and IMR-90. But once the organoids are formed, they combine both of the upregulated genes of each individual cell type. Additional genes are original and not abundantly present in the input cells. This shows that the organoids are more than the sum of the characteristics of the initial input of cells used at the beginning of the process.

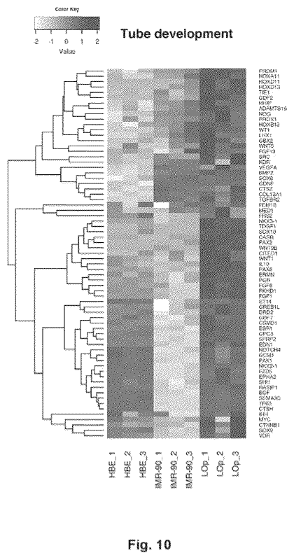

These results suggest that the organoid constitutes a more heterogeneous respiratory system model than HBE or fibroblasts alone. Similarly, all genes associated with tube development were upregulated within the organoid versus cell monolayers (FIG. 10). This suggests that the tubular morphology of the organoids may be a consequence of well orchestrated and complex processes similar to those involved during embryonic lung development. This observation is also supported by trends for other related terms such as alveolar development (FIG. 9), branching morphogenesis, tube morphogenesis, and morphogenesis of branching structure.

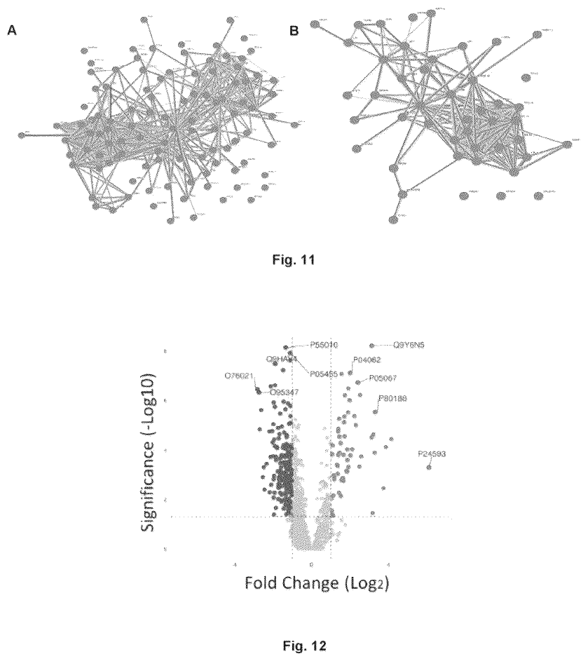

To gain further insight into cellular dynamics, a proteomic analysis was performed using Tandem Mass Tags (TMT). Both types of organoids (LOp and LOi) are reproducible. Three samples (n=3) of each type were used for the analysis. A number 1361 proteins were shared among these samples. A total of 180 proteins were found only in LOp and not in the cell monolayers. A list of upregulated proteins was determined. These proteins were analyzed using the STRING software to collect information from multiple sources, including text mining data from the literature, databases of interaction experiments and complex pathways. Only nodes and networks with high strength (p<0.05) and low false discovery (≤0.001) rate were considered. 122 proteins were specific to the respiratory system, 18 were specific to the throat, and 105 proteins were specific to lung tissue as shown in FIG. 11A. These proteins form interconnected networks, facilitating coordinated communication and regulation of essential cellular functions. Operating within networks enables signal amplification, pathway regulation, and adaptation to changing conditions more effectively than if they were isolated. Interestingly a subset of 46 proteins were associated with the immune system (FIG. 11B), and 33 were associated with response to stress, including chemical and oxidative stress, as well as response to external stimulus.

Wingless related-integrated site (Wnt) was found to be upregulated in LOp, including 12 proteins from the non-canonical pathway and 28 proteins from the signaling pathway. Wnt has been found to regulate epithelial-mesenchymal interactions during lung development. Notably, wingless related-integrated site typically requires supplementation by media for organoid formation. Here the co-culture of epithelial cells and fibroblasts alone provided sufficient signaling and growth factors without need for additional processing. Additionally, gene ontology (GO) analysis was conducted on the upregulated proteins of LOp. The LOp proteins that were downregulated in comparison to HBE were analyzed using STRING and found to be associated with cellular and aerobic respiration, as well as mitochondrial ATP synthesis. The proteomes from organoids derived from immortal cell lines and cell monolayers were compared. 1357 proteins were shared between all samples. A significant number of 180 proteins were found to be exclusive to the organoids and were not identified in the cell monolayers. Using STRING it was found that proteins upregulated in LOi were specific to the lung and the immune system. These two functions are valuable since the models aim to be used to model lung infections in vitro. The associated biological functions included humoral immune response, antimicrobial humoral response, immune system process, viral process, and innate immune response. Surfactant protein A stands out, as it plays an important role in the host-defense mechanisms of the lung, including opsonizing virus, bacteria, and apoptotic cells; it also modulates the production of cytokines and mediators of the inflammatory response. Similar to the upregulated proteins in LOp, the upregulated proteins in LOi also interact with each other, forming interconnected networks that facilitate coordinated communication and regulation of essential cellular functions. Working within these networks allows for more effective signal amplification, pathway regulation, and adaptation to changing conditions than if the proteins were isolated.

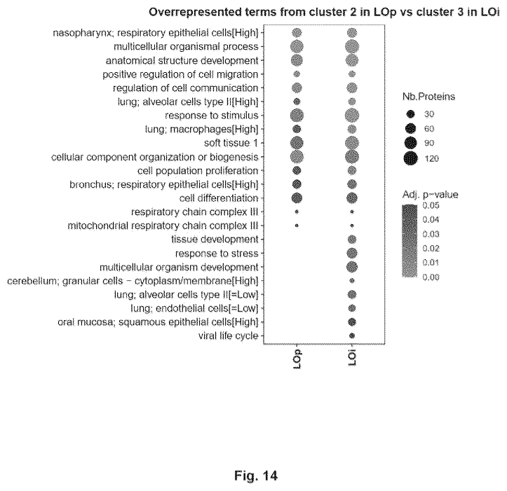

Gene ontology (GO) analysis of upregulated proteins in LOi revealed interesting biological functions. The biological functions associated with the upregulated proteins in LOi are tissue development, biogenesis, cell differentiation, and response to stress. This analysis also revealed an extensive pool of proteins specific to the lung such as lung macrophages, lung AT2 cells, respiratory epithelial cells, and lung mucosa. The presence of autophagy in the organoids, suggests a potential connection to proteins associated with AT2 cells (FIG. 14). Conversely, the cell lines CFBE and IMR-90 have a broad collection of proteins without lung specificity, likely responsible for maintaining housekeeping functions. These proteins were downregulated in LOi. FIG. 14 presents a summary of overrepresented terms, categorizing proteins based on their biological roles or properties in LOp. The figure illustrates the number of proteins corresponding to each term and their statistical significance. Many proteins are associated with lung tissue, including lung AT2 cells, lung macrophages, and bronchus and respiratory epithelial cells. Additionally, proteins involved in cell differentiation, biogenesis, structural development, and cell migration are represented. the proteins upregulated in the organoid are not randomly selected; rather, they play key roles in essential functionalities crucial for lung development and function. Together, these findings highlight the targeted and purposeful nature of the protein expression profile observed in the organoid.

Clearly, the proteomic profiles of organoids derived from both cell lines and primary cells are more specifically related to the lung and respiratory system than the cell monolayers from which they are derived. The organoids also display an upregulation of proteins indicative of tissue development, innate immune response, response to stimulus, cell differentiation, and viral processes.

Significant differences were observed between the proteomes of both types of organoids, LOp and LOi. Upregulated and downregulated proteins in LOp are shown in FIG. 12. Identified using STRING, the biological function and specificity of 102 proteins that were upregulated in LOp relative to LOi were specific to the lung and respiratory system. They were associated with tissue development, multicellular organismal process, response to stimulus, cell differentiation, anatomic structure morphogenesis, and viral entry into host cells. Conversely, proteins that were upregulated in LOi in respect to LOp displayed relatively less specific biological functions; they were mostly associated with changes at the DNA level such as DNA repair, DNA metabolic process, and DNA conformational change. Only 10 proteins were specific to the lung.

Both proteomic profiles were then compared with that of adult lung tissue from three male donors aged 42, 51, and 66 years. Males of this age were chosen because of their susceptibility to COVID-19. Tissue from both superior and inferior regions was used to account for any potential differences in protein levels between the two regions.

The proteome of these samples was clustered into six groups. Cluster 1 demonstrates the similarity of proteins between lung tissues and LOp. Cluster 2 illustrates the similarity of proteins in LOi with those in lung tissues. Cluster 3 indicates the upregulation of proteins in lung tissues compared to the organoids. Cluster 4 shows the upregulation of proteins in LOi compared to those in lung tissue. Clusters 5 and 6 represent the proteins upregulated in both organoids compared to lung tissues.

The proteomic profile of LOp has similarities with the one of adult lung tissue. It shows resemblance to both lung regions: superior and inferior. To reiterate, the primary cells are taken from proximal tissues. There is a benefit for the future development of the organoids, to allow easier and safer collection of cells from proximal regions of the tissues (throat and bronchi) to reproduce the proteome of further distal tissue, itself harder and riskier to collect.

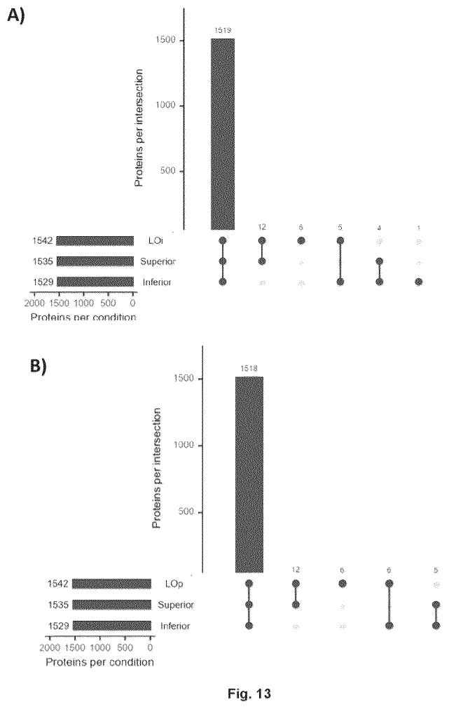

A numerical analysis was performed to quantify differences between all samples (FIG. 13). As shown in FIG. 13A, adult lung tissue and LOp shared 1518 proteins, from which 5 were exclusively found in the tissues, and 6 in the organoids. Adult lung tissues and LOi shared 1519 proteins, with only 4 proteins absent from the organoids (FIG. 13B).

Gene ontology (GO) analysis was conducted on clusters 1, 3, and 6 of the proteome of adult lung tissues and organoids. These clusters correspond to sections of the proteome representing similarities between LOp and tissues, upregulated proteins in the tissues, and upregulated proteins in the organoids, respectively. The overrepresented terms in cluster 1 show that the proteins in LOp and adult tissues are related to alveolar type II cells, inflammatory response, lung macrophages, basal layer, epithelial migration, and response to stress. The proteins upregulated in lung tissues in respect to organoids (cluster 3) show overrepresented terms related to goblet cells, indicating a higher number of these cells in the tissue. Lung epithelial and lung cell differentiation, as well as proteins related to secretory cells, were also found to be functionalities of the upregulated proteins in the tissue. On the other hand, the functionalities of the proteins upregulated in the organoids in respect to adult lung tissues (cluster 6) are related to bronchus respiratory epithelium, oral mucosa, and nasopharynx epithelium. This suggests that some of the proteins of the organoids correspond to the proximal lung region. Additionally, several proteins related to viruses were found in the organoids, such as coronavirus disease, virus replication, viral transmission, and response to virus, highlighting the potential application of the model for the study of viral diseases. Proteins associated with endothelial cells, alveolar cells, and lung macrophages were also found in this cluster.

In summary, the proteome of organoids was found to be much more similar to that of adult lung tissues than the cell monolayers. Only 4 or 5 proteins are found exclusively in the tissue and not in the LOi or LOp, respectively. In terms of regulation, the similarity was strongest for organoids derived from primary cell lines, as could be expected from their increased differentiation capacity.

Lung organoids derived from primary cells (LOp) offer many advantages, such as elevated levels of innate immune response proteins, an enriched pool of lung and respiratory system-specific proteins, and clear differentiation into multiple cell types. But the lung organoids derived by immortalized cell lines (LOi) remain potentially useful as they also present multiple enriched pathway specific to the lung and respiratory system, albeit less than their LOp counter parts. Their morphology is also tubular. Their use would circumvent the need for primary cells, which are more expensive and challenging to acquire and culture than cell lines.

Gene ontology (GO) analysis was conducted accordingly on the upregulated proteins of LOp. FIG. 14 presents a summary of overrepresented terms, categorizing proteins based on their biological roles or properties in LOp. FIG. 14 illustrates the number of proteins corresponding to each term and their statistical significance. The terms depicted offer insight into the varied biological roles and properties of the proteins identified in LOp. Many proteins are associated with lung tissue, including lung AT2 cells, lung macrophages, and bronchus and respiratory epithelial cells. Additionally, proteins involved in cell differentiation, biogenesis, structural development, and cell migration are represented. the proteins upregulated in the organoid are not randomly selected; rather, they play key roles in essential functionalities crucial for lung development and function. Together, these findings highlight the targeted and purposeful nature of the protein expression profile observed in the organoid.

Gene ontology (GO) analysis of upregulated proteins in LOi revealed interesting biological functions. FIG. 14 provides an overview of terms that are overrepresented, organizing proteins according to their biological roles or properties in LOi. It shows the number of proteins associated with each term and their statistical significance. The biological functions associated with the upregulated proteins in LOi are tissue development, biogenesis, cell differentiation, and response to stress. This analysis also revealed an extensive pool of proteins specific to the lung such as lung macrophages, lung AT2 cells, respiratory epithelial cells, and lung mucosa.

Clearly, the proteomic profiles of organoids derived from both cell lines and primary cells are more specifically related to the lung and respiratory system than the cell monolayers from which they are derived. The organoids also display an upregulation of proteins indicative of tissue development, innate immune response, response to stimulus, cell differentiation, and viral processes.

Given the over expression of proteins associated with viral transcription, and viral process found on the organoids, specific genes relevant for adult viral infections were investigated.

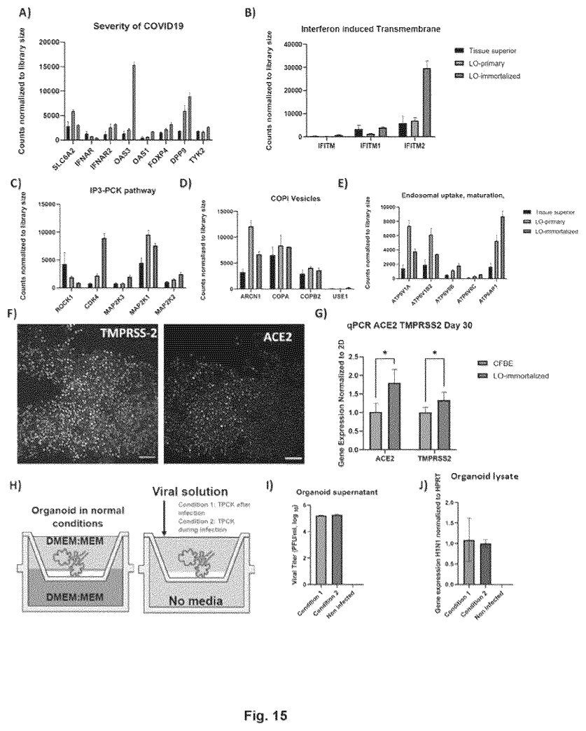

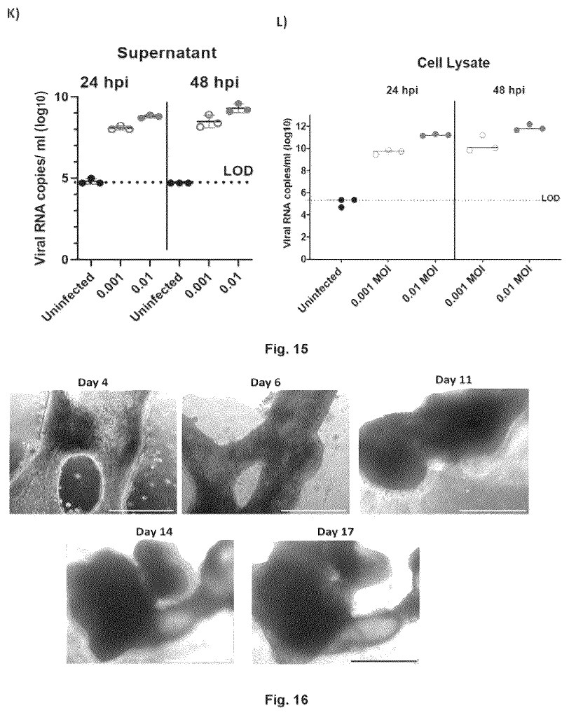

RNA sequence revealed that lung tissue and both organoids have genes associated with the severity of COVID-19. Interestingly, these genes are up regulated on the organoids suggesting an exacerbated condition, due to the inflammatory response of fibroblasts used for the generation of LO. FIG. 15A shows all the genes associated with the severity of COVID-19 on tissue and organoids. These genes are: Solute Carrier Family 6 Member 2 (SLC6A2), Interferon Alpha and Beta Receptor Subunit 1 (IFNAR1), Interferon Alpha And Beta Receptor Subunit 2 (IFNAR2), 2′-5′-Oligoadenylate Synthetase 3 (OAS3), 2′-5′-Oligoadenylate Synthetase 1 (OAS1), Forkhead Box P4 (FOXP4), Dipeptidyl Peptidase 9 (DPP9), Tyrosine Kinase 2 (TYK2). The highest expression of these genes was found in LO derived from immortalized cell lines, except for IFNAR1, which expression decreased on the organoids. Low expression of interferon receptors increases the odds of critical illness due to COVID-19. The most abundant gene found in LOi was OAS3, this is an antiviral enzyme which plays a role in cellular innate antiviral response, when it is activated, it inhibits the protein synthesis thus terminating viral replication (NIH, 2023). OAS3 is found abundantly in both CFBE and IMR-90 cell lines, and as it was expected it increased when the organoid was formed.

Interferon induced transmembrane genes were also found in tissue and organoids. These genes restrict cellular entry of diverse viral pathogens such as influenza A, COVID-19, and Ebola virus. Interferon Induced Transmembrane 2 (IFITM2) was the most abundant transmembrane in all the samples, as it shows in FIG. 15B. It also shows a significate increase in LO derived by immortalized cell lines. These transmembrane are also abundant on IMR-90 cells.

In addition, using RNA sequence, human host factors required for influenza infection were investigated in the organoids and lung tissue by different pathways. Genes involved in Phosphoinositide 3-kinase-Protein kinase C (IP3-PCK) pathway were investigated. These genes are Rho Associated Coiled-Coil Containing Protein Kinase (ROCK1), Cyclin Dependent Kinase 4 (CDK4), Mitogen-Activated Protein Kinase 3 (MAPK3), Mitogen-Activated Protein Kinase 1 (MAP2K1), Mitogen-Activated Protein Kinase 2 (MAP2K2). As it is shown if FIG. 15C all these genes, except for ROCK1 were upregulated in the organoid in comparison with the tissue. These genes help for viral entry and enhances viral replication.

Coat protein complex I (COPI) was also investigated. This mechanism involves genes associated with the entry of virus by endosomal trafficking. The investigated genes are Archain 1 (ARCN1), COPI Coat Complex Subunit Alpha (COPA), COPI Coat Complex Subunit Beta 2 (COPB2), Unconventional SNARE In the ER 1 (USE1). These genes were upregulated on the organoids, in comparison to the tissue, and they were more abundantly in LOp as shown in FIG. 15D. Endosomal uptake, maturation, acidification and fusion genes were also investigated mainly for their capacity to acidify intracellular organelles allowing the viral entry. FIG. 15E, illustrates these genes, including ATPase H+ Transporting V1 Subunit A (ATP6V1A), ATPase H+Transporting V1 Subunit B2 (ATP6V1B2), ATPase H+ Transporting V0 Subunit B (ATP6V0B), ATPase H+ Transporting V0 Subunit C (ATP6V0C), ATPase H+ Transporting Accessory Protein 1 (ATP6AP1). These genes were up regulated in Lung organoids derived by primary and immortalized cell lines.

Angiotensin-Converting-Enzyme 2 (ACE2) was also investigated, since it is considered the main mechanism for entry of SARS-CoV-2. Transmembrane serine protease 2 (TMPRSS2) was also studied, since it is involved in the priming of SARS-CoV-2 and influenza viruses including H1N1, and other coronaviruses. FIG. 15F shows the expression of both proteins during the organoid formation, at day 7. Interestingly TMPRSS2 is located both in the periphery and in the core of the organoids, which may facilitate the virus infection without needing to disrupt the 3D structure of the organoid as is often required. Quantitative PCR was used to quantify the expression of both proteins in the mature organoids as well as comparing their expression to that found in CFBE monocultures grown at the ALI. FIG. 15G shows a significantly higher expression in the organoids than in the ALI cultures. This confirms that the disclosed organoid expresses necessary proteins to promote influenza viral infection and highlights the potential of the model for studying other viral diseases in human lungs. Therefore, as a proof of concept, the organoids were infected using influenza H1N1-A/R/8/34 with a multiplicity of infection of 1 for 1 hour using two different conditions, (FIG. 15H). In condition 1, the post infection media contain trypsin Tosyl phenylalanyl chloromethyl ketone (TPCK), while in condition 2, the infection was done along with TPCK supplementation. The organoid lysates and supernatants (n=2) were analyzed 48 h post-infection using plaque assays to obtain viral titer (FIG. 15I). There was no significant difference between the two conditions. The infection was evident, reaching a PFU of 5×10⁷. The organoid lysates were analyzed using qPCR to identify the viral gene copy of influenza H1N1. Robust expression of Nucleocapsid Protein (NP) was detected under both conditions of H1N1 infection while no expression was observed in negative control cells, demonstrating successful infection of H1N1 of the organoids (FIG. 15J). Organoids were also infected with SARS-COV-2 during 24 h and 48 h with two different MOI: 0.01, and 0.001. In both cases, viral copies were found in the organoid supernatant and organoid lysate post-infection with significant difference between dosages (FIGS. 15K and L).

Organoids derived from primary airway epithelial cells of a donor coded as BA00814, 67 year old female subject with asthma, were generated. It is encompassed that organoids can be made as described herein with healthy and unhealthy donors. Firstly, the pre-established protocol discussed was used. But no organoid could be formed after 7 days of culture. Instead, cells remained rounded and dispersed within the gel. Given the role of fibroblasts promoting chronic inflammation in already inflammatory diseases, the original density of fibroblasts was modulated of 2.5×10\hat{ }6/ml to 1.6×10\hat{ }6/ml. No organoid was formed. Therefore, additional conditions were tested. When culturing 5×10\hat{ }6 per/ml of airway epithelial cells with 5×10\hat{ }5 per/ml of fibroblasts, at a final ratio 10:1, at day 6 and day 14, under this condition, some cells regrouped, but the typical organoid achieved with healthy cells was not formed. At day 14 the cells looked clustered together without a specific morphology, and cells shedding from the “main” structure were observed. Therefore, these conditions were not considered successful. Subsequently, airway epithelial cells were cultured without fibroblasts, on top of Geltrex. At day 3, it was observed cells went into a more tubular morphology with bifurcations. However, these structures started to disassociate, some at day 6, while some only at day 11. All structures were disassociated at day 14, into more sphere like structures. Culturing asthmatic airway epithelial cells without fibroblasts lead to better morphology but just for short term culture. It has been well documented that during asthma there is interleukin storm associated with inflammation. IL-4, IL-5, and IL-13 are highly abundant, while low levels of IL-10 are normally found in patients with asthma. Steroid treatment are commonly used for asthma increases IL-10 production. Therefore, asthmatic airway epithelial cells were cultured, without fibroblasts, in a medium with 10 ng of IL-10. This condition was found to be optimal. Organoids grew, starting to show a robust and tubular morphology at day 4, with continued growth without disruption until day 17. From day 14 to day 17, there was not notorious change of morphology. The organoids formed under this condition are shown in FIG. 16.

All this data suggest the usefulness of the model to study respiratory viral infections, as well as potential therapeutic drugs against them.

A new lung-organoid type model is thus provided. The model resembles the tubular and arborized architecture of the lungs, while integrating some of their proximal and distal components in one single model. To improve cell accessibility, airway epithelial cells from the proximal airways (Trachea/Bronchi) and, alternatively, immortalized bronchial epithelial cells (CFBE) were used instead of deep lung tissue biopsies. These models are fast-maturing and show an adult phenotype, two further sought-after characteristics. The value of these new lung organoid models is demonstrated by their use in modelling infections of important pathogens (COVID19, Influenza H3N2, H1N1) and the prevalent asthmatic condition.

It is thus provided a novel approach to induce the formation of human lower respiratory tract organoids, which demonstrate physiological lung morphogenesis and express multiple cellular populations of the proximal and distal regions of the lung. The organoids growth gives rise to goblet, basal and ciliated cells, as well as surfactant proteins of the alveolar region, produced by alveolar type II cells. The resulting model provides a more physiological relevant model of the lung, which include cell interactions and replicates their functions in vivo. The model started with two different cell types: airway epithelium and fibroblasts. Their interaction triggers the tube morphogenesis by epithelial to mesenchymal transition to further differentiate into multiple sub-type of cells observed in the organoid. This sophisticated morphology has a macro-scale dimension. Such structures can be fabricated using bioprinting but it has never previously been created directly from cellular interactions themselves.

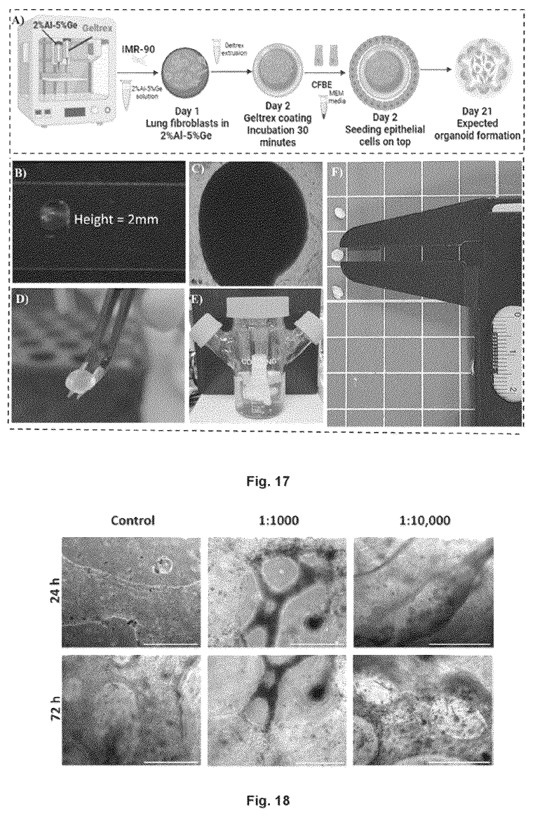

Bioprinting was explored to automate the fabrication of lung organoids to reduce costs and reduce variability due to manipulations (see FIG. 17). The goal is to improve the robustness of the model and ease their handling. Biofabricated models can easily be transported to a spinning bioreactor. The use of spinning bioreactors for culturing organoids enhances oxygen and nutrients absorption, allowing preservation over a longer time.

The LO generated as escribed herein can be used until day 30. Afterwards, some degree of disintegration of the organoid is observed. Manipulation of the organoid grown in transwells is difficult, hampering potential high throughput characterizations. A 3D bioprinter (BioX Cellink) was used to extrude AlGe containing lung fibroblasts solution into 96-well plates. A “Droplet” configuration was used while extruding the solution. On the second day, a layer of geltrex was used to coat the droplets. After polymerization, airway epithelial cells, CFBE, were added on the top of the gel.

Three different densities were tested. During manual fabrication every organoid had an initial cell density of 250,000 cells deposited in 100 μl of AlGe. To keep the cell original density, OG, per organoid, 10 million IMR-90 cells per ml were used in bioprinting. Every droplet included 25 μl of solution, and approximately 250, 000 cells. To reduce the cell concentration, solutions were diluted 100 times, 0.01 OG, or 10 times, 0.1 OG.

The diameter of the droplets was measured using widefield microscopy. It was observed that the droplets shrunk over time regardless of the cell density. They were robust and maintained their shape beyond 30 days of culture. The thickness of the droplets was 2 mm, much greater than that of 100 μm, achieved by manual methods, thereby improving their cohesiveness for handling. The 0.01 OG cell concentration was used for the deposition of Geltrex and CFBE.

The resulting lung ‘organoids’ were kept for more than 30 days in static conditions, without evident disassociation. Organoids could be handled with tweezers and cultured them into a spinning bioreactor over 21 days with no signs of damage. Based on the preliminary characterisation results, automated biofabrication has the potential to achieve a faster fabrication rate (35 seconds per droplet), higher reproducibility (in terms of size), a higher flexibility in varying cell density, and greater organoids robustness.

Regardless of their complexity, the organoids reached maturation after 21 days of culture, which is faster than existing methods. The present protocol can induce the formation of organoids with cell lines, LOi, and primary cells LOp. Organoids derived by immortalized cell lines, LOi, showed superiority in comparison to cell monolayers. A high number of proteins with specificity from the lung, tissue development, and innate immune system were upregulated. Similarly, patient-derived organoids, LOp, showed an enriched pool of lung specific proteins, innate immunity, and response to stress which resemblance closely the proteins found in lung tissue. While LOi remains advantageous as it circumvents the need for primary cells, LOp can be used where personalized treatment or analysis are needed and to achieve better cell differentiation. Both types of organoids demonstrate clinical relevance as they mimic the phenotype of adult lung tissues, and thus can serve as effective models for studying respiratory diseases, such as viral infections. As a proof of concept, LOi can be infected with influenza H1N1, and SARS-COV-2, opening the door for modeling other infection diseases, and other influenza type A viruses, with similar viral tropism that the one which is observed in the human body.

Also encompassed is a microfluidic based model of the lower airway epithelium for the testing of anti-inflammatory drugs. The device encompassed features an air-liquid interface, three types of cells (epithelial, fibroblasts, and endothelial cells), basement proteins, a screen, and channels for air flow and for irrigation of the cells with cell culture medium. The present disclosure is a novel model of the respiratory tract with airway mucosa that accurately reproduces the immune response in vivo. Three PDMS-based layers are cast on 3D-printed mold and assembled to form the microfluidic device. Alginate-gelatin hydrogel laden with fibroblasts can be cast or bio-printed and transferred to the center chamber of the microfluidic device for 3D cell culture while providing lung-mimetic stiffness. The endothelial channel is then coated with lung endothelial cells. After that, the central chamber (the top of the fibroblasts-laden alginate-gelatine hydrogel) is coated with basement membrane proteins followed by seeding primary human bronchial epithelial cells. The membrane coating is to promote cell adhesion and lung branching morphogenesis to form an arborized network. The airway epithelium channel is perfused with air to form an air-liquid interface and ensure differentiation of AT2-expressing epithelial cells.

It was shown that the organoids can be infected with influenza H1N1, H3N2 and SARS-CoV-2. Viral replication was observed in all infections and confirmed in organoid lysate in case of SARS-CoV-2 and influenza H1N1. Viral titer was found to be within the range of previous published work using organoids and cell monolayers for both influenza65 and SARS-CoV-2. Accordingly, it is encompassed the use of the organoid, device and/or the kit encompassed herein to characterize a respiratory viral infection and/or measure therapeutic drug activity against the respiratory viral infection. The viral infection can be from, but not limited to, influenza A, COVID-19, Nipah virus, or Ebola virus. to measure therapeutic drug activity against the respiratory viral infection.

The original protocol for organoid formation was adapted for using cells from a patient with asthma. The incorporation of fibroblasts in typical concentrations was not beneficial for organoid formation. A lower fibroblast concentration improved the outcome. Organoid formation could not be induced without removing the fibroblasts from the culture. This observation is interesting because it highlights the essential role of fibroblasts in supporting organoid growth under healthy conditions, whereas under unhealthy conditions, fibroblasts may contribute to microenvironmental damage, inflammation, and impaired remodeling.

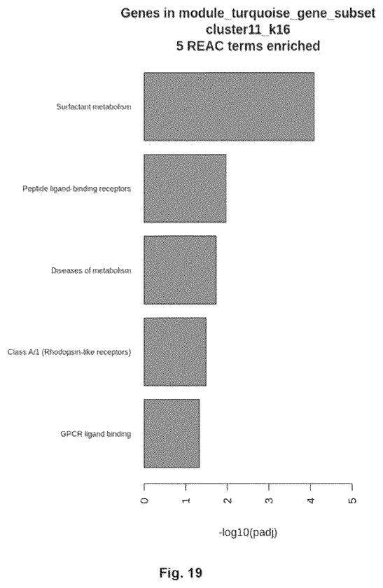

It is encompassed that other non-epithelial cell types could be incorporated in lung organoid models, for instance, macrophages. Lung macrophages are distributed in proximal and distal airways of the lung. They are part of the innate immunity of the lung. They are critical to maintain lung homeostasis, and clear pathogens. Their incorporation could be beneficial and yield a better understanding of the pathophysiology of respiratory infections. In an embodiment, the integration of monocytes (THP-1) is encompassed, as shown in FIG. 18. A cell density of 1:1000 was used, and 1:10,000 in relationship to airway epithelial cells. The preliminary study showed a potential strategy for the integration of different cell types into organoids. Monocytes were cultured on top of an 8 μm porous membrane for 24 or 72 h on day 4 of organoid formation. Increasing time and cell density caused partial breakdown of the organoid.

When a quantitative comparison was conducted between organoids generated using the protocol outlined herein, focusing specifically on lung organoids (LOp), and alveolar organoids (alveolospheres) previously described in a publication by Chiu (2022, Cell Discovery, 8: 57), the study revealed that both types of organoids shared similar gene expression profiles, particularly in genes related to surfactant metabolism, as analyzed through Gene Profile (G. profile) analysis, as depicted in FIG. 19. It was notable that LOp organoids and alveolospheres demonstrated an abundance of genes associated with surfactant metabolism, indicating their differentiation to distal regions of the lung. This differentiation was observed regardless of the proximal origin of the primary cells used (trachea/bronchus), affirming the versatility of LOp organoids in representing distal lung regions.

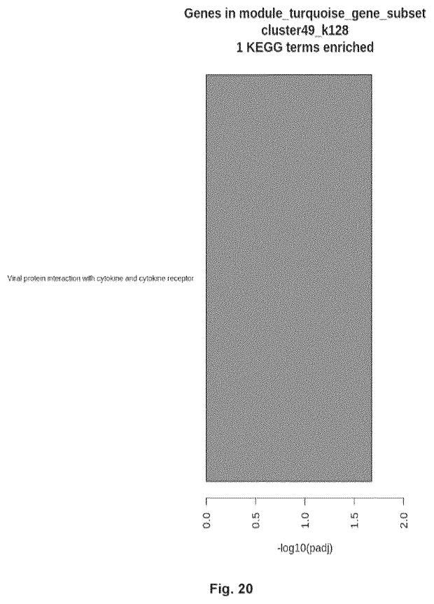

Furthermore, the analysis revealed that LOp organoids exhibited higher expression levels of genes associated with viral protein interaction with cytokines and cytokine receptors compared to alveolar organoids, as illustrated in FIG. 20. This suggests their potential superiority in modeling viral infections compared to other organoid models.

### EXAMPLE I

### Experimental Procedures

### Cell Culture and CFBE in Air-Liquid Interface

CFBE and IMR-90 were cultured in Eagle's minimum essential medium (VWR, CA45000-380) supplemented with 10% (vol/vol) FBS (Sigma-Aldrich, F1051), 2 mM L-glutamine (Thermo Fisher, A2916801), 1% Penicillin Streptomycin (Fisher Scientific, SV30010), and incubated at 37° C. in 5% CO₂-95% O₂. Human Vocal Fold Fibroblasts (hVFF) were cultured in Dulbecco's Modified Eagle's Medium (Life Technologies, 12430054) supplemented with 10% (vol/vol) FBS (Sigma-Aldrich, F1051), 1% Aminoacids solution (Thermo Fisher, 11130051), 1% Penicillin Streptomycin (Fisher Scientific, SV30010), and incubated at 37° C. in 5% CO₂-95% O₂. For establishment of ALI, CFBE were seeded on fibronectin (Promo cell, C-43060) coated permeable cell culture inserts (Fisher Scientific, 08-771-10) and cultured for 21 days before the study.

### Primary Airway Epithelium Culture in Air-Liquid Interface

Primary human bronchial epithelial were provided by Primary Airway Cell Biobank (PACB) in the Cystic Fibrosis Translational Research Centre at McGill University. Cell from male and female donors of 51 and 53 years old, respectively were used. Cells were seeded on collagen IV coated cell culture inserts (Fisher Scientific, 08-771-10) and cultured in ALI medium provided by PACB. After 3 days, the apical medium is removed, and cells were allowed to differentiate for 28 days at the air-liquid interface before study. The medium in the basolateral chamber is changed every two days. Once a week, the apical mucus is aspirated carefully to avoid disruption of the monolayer. Followed by washing the apical side with PBS without Ca²⁺.

### Establishing Adult CFBE-Derived Human Lung Organoids

Human fetal lung, IMR90, cells were cultured overnight in different alginate-gelatin hydrogels in a density of 250,000 cells per/cm² using DMEM (Corning, 15-017-CV) with Penicillin-Streptomycin (Fisher Scientific, SV30010). Non-essential aminoacids (Fisher Scientific, LS11140050) and FBS (Sigma-Aldrich, F1051) at 37° C. The media was removed, and the hydrogels were coated using 100 μl/cm² of Geltrex (Thermo Fisher, A1413202) during 30 minutes at 37° C. Afterwards Human Bronchial CF Epithelial cell line was seeded on the top in a density of 500,000 cells per/cm² using EMEM (Corning) with L-Glutamine (Thermo Fisher, A2916801) forming a co-culture interface. The next day the medium was substituted by a combination in a ration 1:1 of DMEM and EMEM supplemented with 3 mM CaCl₂ (Sigma, Aldrich, C4901). The organoids were formed by the cellular and extracellular matrix interactions. After 5 days of culture, the maturation, and branching of the organoids was observed, and the organoids were cultured in air-liquid interface until day 30 at 37° C. under a humidified atmosphere with 5% CO₂ and 95% air. During culturing, medium was refreshed at most every two days.

### Immunofluorescence Staining

The morphological analysis of the organoids was made at day 7, 16, 21, 30 and 40 days, according to the study. Organoids were washed with PBS (Fisher Scientific, 10-010-049) supplemented with 10 mM CaCl₂ (Sigma, Aldrich, C4901) three times for 3 minutes. Afterwards, the organoids were fixed with 3.7% paraformaldehyde, permeabilized with 0.3% Triton X-100 (Sigma Aldrich, T9284), and blocked for 2 hours with 10% Goat serum (Thermo Fisher, 50062Z) at room temperature. E-cadherin monoclonal antibody (Fisher Scientific, MA5-11495) and ZO-1 (Thermo Fisher, 61-7300) were used like primary antibodies diluted in 10% goat serum at 4° C. overnight followed by incubation of secondary antibodies Rabbit anti-mouse IgG H&L Alexa Fluor 555 (Abcam, ab150126) and Goat-anti Rabbit IgG Alexa Fluor 488 (Fisher Scientific, A11034), respectively, for 2 hours at room temperature. Nuclei and actin filaments were counterstained with Hoechst dye (Thermo Fisher H3569) and Phalloidin-647 (Fisher Scientific, A22287) respectively. The confocal images were acquired using LSM-710 inverted confocal microscope.

### Protein Expression

For evaluating the expression of proteins related to infection and replication of influenza H1N1 and SARS-COV-2 such as Transmembrane Protease Serine 2, TMPRSS, and Angiotensin Converting Enzyme 2, ACE2, primary antibodies were used and incubated overnight in 10% goat serum followed by incubation of secondary antibody Goat-anti Rabbit IgG Alexa Fluor 488 (Fisher Scientific, A11034) for 2 hours at room temperature. The primary antibodies used were the ACE2 recombinant rabbit monoclonal antibody (SN0754) from ThermoFisher, and Recombinant Anti-TMPRSS-2 antibody (AB109131) from Abcam.

### Multiple Cell Populations

For evaluating the expression of proteins associated with distal and proximal markers of the LRT, antibodies conjugated with Alexa were used and incubated overnight in 10% goat serum followed by Hoechst dye incubation for 5 minutes. Surfactant protein B (Santa Cruz-133143-AF488) and Surfactant protein C (Santa Cruz, 518029, AF594) were used as markers of alveolar type II cells. Podoplanin (Santa Cruz, 376695, AF488) and HOPX (Santa Cruz, 398703-AF647) were used as markers of alveolar type I cells. Cytokeratin 14 (Santa Cruz, 53253-AF594) was used as marker of basal cells. Mucins: MUC5B (Santa Cruz, 21768-AF488) and MUC5C (Santa Cruz, 21768-AF680) were used as markers of goblet cells. B-Tubulin (Santa Cruz, 166729-AF488) was used as marker of ciliated cells.

### RNA Isolation and Quantitative PCR

RNA was extracted using Illustra RNA Spin Mini kit (Ge Healthcare, GE25-0500-71) according to the manufacturer's instructions. RNA (500 ng) was reverse transcribed with 5×RT Master Mix (Diamed, ABMG486) with incubation at 25° C. for 10 minutes, 42° C. for 1 h, and 85° C. for 5 minutes. Quantitative PCR (qPCR) was performed using Power track SYBR green (ThermoFisher, A46109). The reaction was performed using a QuantStudio 7 Flex Real-Time PCR system and the following protocol: 20 s at 95° C. and 40 cycles at 95° C. (1 s) and 60° C. (20 s). Change in gene expression relative to housekeeping gene (ΔΔCt) was calculated using the manufacturer's software package. Primers were predesigned for IDT, except for the house keeping gene HPRT, ACE2, and TMPRSS2. They are listed in key Resources Table II.

### Bulk RNA Sequencing and Analysis

The extraction, library preparation and sequencing services were provided by the Génome Québec Centre d'expertise et de services.

### Library Preparation

Ribosomal RNA were depleted from 250 ng of total RNA using QIAseq FastSelect. cDNA synthesis was achieved with the NEBNext RNA First Strand Synthesis and NEBNext Ultra Directional RNA Second Strand Synthesis Modules (New England BioLabs). The remaining steps of library preparation were done using and the NEBNext Ultra II DNA Library Prep Kit for Illumina (New England BioLabs). Adapters and PCR primers were purchased from New England BioLabs.

Libraries were quantified using the KAPA Library Quantification Kits—Complete kit (Universal) (Kapa Biosystems). Average size fragment was determined using a LabChip GX II (PerkinElmer) instrument.

The libraries were normalized and pooled and then denatured in 0.02N NaOH and neutralized using HT1 buffer. The pool was loaded at 175 pM on a Illumina NovaSeq S4 lane using Xp protocol as per the manufacturer's recommendations. The run was performed for 2×100 cycles (paired-end mode). A phiX library was used as a control and mixed with libraries at 1% level. Base calling was performed with RTA v3. Program bcl2fastq2 v2.20 was then used to demultiplex samples and generate fastq reads.

### Sample Preparation

Cells monolayers and organoids were washed with cold PBS (×3) following by incubation on ice cold RIPA buffer (Sigma, R0278). The cell monolayers and organoids are broken using a plastic cell scraper. Organoids are passed by 21 G needle until completely disassociated. Cells are collected and centrifuged at 16000 G for 50 minutes at 4° C. The supernatants are collected, protein quantification was performed with Qubit Protein Assay (ThermoFisher, Q33211), and normalized to 45 ng of protein. Samples were stored at −80° C. until their analysis. All the reported proteins have 95% of threshold.

Samples were treated with TMT-16plex reagents (ThermoFisher Scientific) according to the manufacturer's instructions. Labelled peptides were fractionated using Pierce™ High pH Reversed-Phase Peptide Fractionation Kit into 8 fractions. Each fraction was re-solubilized in 0.1% aqueous formic acid and 2 micrograms of each was loaded onto a Thermo Acclaim Pepmap (Thermo, 75 uM ID×2 cm C18 3 μM beads) precolumn and then onto an Acclaim Pepmap Easyspray (Thermo, 75 uM×15 cm with 2 uM C18 beads) analytical column separation using a Dionex Ultimate 3000 uHPLC at 250 nl/min with a gradient of 2-35% organic (0.1% formic acid in acetonitrile) over three hours running the default settings for MS3-level SPS TMT quantitation (McAlister et al, 2014—Anal Chem. 2014 Jul. 15; 86(14):7150-8. doi: 10.1021/ac502040v.), on an Orbitrap Fusion instrument (ThermoFisher Scientific) was operated in DDA-MS3 mode. Briefly, MS1 scans were collected at 120,000 resolution, scanning from 375-1500 m/z, collecting ions for 50 ms or until the AGC target of 4e5 was reached. Precursors with a charge state of 2-5 were included for MS2 analysis, which were isolated with an isolation window of 0.7 m/z. Ions were collected for up to 50 ms or until an AGC target value of 1e4 was reached and fragmented using CID at 35% energy; these were then read out on the linear ion trap in rapid mode. Subsequently, the top 10 (height) sequential precursor notches were selected from MS2 spectra for MSquantitative TMT reporter ion analysis, isolated with an m/z window of 2 m/z, and fragmented with HCD at 65% energy. Resulting fragments were read out in the Orbitrap at 60,000 resolutions, with a maximum injection time of 105 ms or until the AGC target value of 1e5 was reached.

### Mass Spectrometry Raw Data Analysis

To translate raw files into protein identifications and TMT reporter ion intensities, Proteome Discoverer 2.2 (ThermoFisher Scientific) was used with the built-in TMT Reporter ion quantification workflows. Default settings were applied, with Trypsin as enzyme specificity. Spectra were matched against the human protein fasta database obtained from Uniprot (2022). Dynamic modifications were set as Oxidation (M), and Acetylation on protein N-termini. Cysteine carbamidomethyl was set as a static modification, together with the TMT tag on both peptide N-termini and K residues. All results were filtered to a 1% FDR.

### Rheological Measurements

The shear moduli were determined using a torsional rheometer with parallel plates (Discovery HR-2). Isothermal time sweeps were applied at a frequency of 0.1 Hz and 0.1% strain at 37° C. The shear moduli were recorded and analyzed using TA instruments software. Experiments were conducted in triplicate.

### Live/Dead Assay

The viability of the organoids was evaluated after 7, 16 and 30 days of culture using a live/dead kit (Fisher Scientific, L3224). The confocal images were acquired using an inverted confocal laser scanning microscope (LSM710, Zeiss). Live cells were shown in green fluorescence and dead cells were shown in red.

### Biomaterial Synthesis

AlGe-A solution of 2% Alginic acid molecular weight (MW) 212.121 g/mol (Sigma, A0682) was prepared in MilliQ water and sterilized with 0.2 μm PES filter (Fisher Scientific, FB12566502). The solution was mixed with 5% sterile Gelatin type A (Sigma, G2500). The hydrogel was formed by the incubation with 100 mM CaCl₂, MW 147.02 g/mol (ACP, C-0360) for 15 minutes.

### RGD Modification

The method was adapted from previous protocols (Hermanson, 2008). Overnight 0.1% of alginate was dissolved in 0.1 MES buffer, 0.5 M NaCl, at pH 6. Next day, 2 mM of EDC and 5 mM NCH (ThermoFisher, 24510) were added into alginate solution, allowing its reaction for 15 minutes. Subsequently, pH was raised to 7.3 and RGD was incorporated, and let to react for 2 hours. 10 mM of Hydroxylamine-HCl was used for quenching the NHS. Dialysis was done for 4 hours, and the solution placed in the freeze-dryer. Fourier-transform infrared spectroscopy was used to ensure RGD conjugation.

### Flow Cytometry

To assess the percentages of the types of cells, the LOs were analyzed by flow cytometry. Briefly, the organoids were dissociated with Trypsin-EDTA (Fisher Scientific, MT25053CI) for 30 minutes at 37° C. Clumps were disassociated with 21 G gauge needle. Cell suspension was collected and centrifuged for 5 minutes. Supernatants were discarded and the cell pellets were wash twice with PBS. Samples were fixed with 4% PFA, and permeabilized with 0.1% Triton X-100. Subsequently, cells were incubated with 10% Goat serum for 15 minutes. The cells were incubated with antibodies conjugated (Key Resources Table) in flow cytometer staining buffer (ThermoFisher, 00-4222-57) for 20 minutes. Beads were incubated with single antibodies, and Fluorescence minus one (FMO) organoid were used to calibrate the instrument. A BD FACS LSRFortessa was used to analyze the samples.

### Influenza H1N1 Infection in Human Organoids

Before infection, the LRT-LO were washed twice with DMEM:MEM without serum. Two different conditions of infection were used. In both conditions organoids were infected with MOI of 0.1 and 1 of influenza H1N1-PR-8 dissolved in serum free medium. In condition 1, after the infection 0.1% trypsin TPCK was added into the medium containing 1% BSA. In condition 2, 0.1% Trypsin TPCK was integrated into the cell medium, and after infection the media was switched by DMEM:MEM with 1% BSA. The virus was incubated for 1 h at 37° C., and every 15 minutes the samples were rocked. At the indicated times, the organoids and cell culture medium were harvested for detection of viral load. The cell-free culture medium was analyzed as “supernatant.”

### Plaque Assay

Plaque assay was performed to determine titers of the virus supernatant samples. In short, MDCK cells were seeded on 6 well plates. Confluent monolayers were inoculated with 400 μl of 10-fold serial dilutions of samples and were incubated 1 h at 37° C. After the inoculum was removed, the monolayers were overlaid with Carboxymethylcellulose (CMC) adding 0.1% of trypsin TPCK and were further incubated for 3 days. The monolayers were fixed with 70% EtOH and crystal violet for 20 minutes. Viral titers were calculated as plaque-forming units per milliliter.

Organoids derived from immortalized cells at 21 days of culture were embedded in resin after sequential fixation in 2.5% glutaraldehyde in 0.1 M cacodylate buffer (pH 7.2) at 4° C. The sections were examined under 2.5 nm at 30 kV Focused Ion Beam Microscope.

### Statistical Analysis

GraphPad was used for data analysis. Two-way ANOVA was performed for evaluation of SARS-CoV-2 infection P<0.05 was consider statistically significant. *p<0.05, **p<0.01, ***p<0.005. For influenza infections, since less parameters were used One-way ANOVA was used. ***p<0.005. T. test was used for the evaluation of differentially expressed genes associated with viral infection, and cell markers as follows Differential expression was performed using DESeq2 R package. Differentially expressed genes were define using adj-pvalue<X and |log 2FCshrink|≥4×leading respectively X, Y, Z DEGs for A vs B, A vs C and B vs C. DEGs heatmap were drawn based on z-score of normalized count. It was considered significant when p. adjust was <0.05″.

### EXAMPLE II

### Generation of Asthmatic Organoids

Human fetal lung, IMR90, cells were cultured overnight in alginate-gelatin hydrogels with different density, according to specific conditions. 250,000, 166,000, and 50,000 cells per/cm² were used using DMEM (Corning, 15-017-CV) with Penicillin-Streptomycin (Fisher Scientific, SV30010) non-essential amino acids (Fisher Scientific, LS11140050) and FBS (Sigma-Aldrich, F1051) at 37° C. The media was removed, and the hydrogels were coated using 100 μl/cm2 of Geltrex (Thermo Fisher, A1413202) for 30 min at 37° C. Afterwards, Human Bronchial Epithelial derived from an asthmatic patient were seeded on the top with a density of 500,000 cells per/cm2 using Bronchial Epithelial Cell Growth Medium (BEGM) acquired from PACB and L-Glutamine (Thermo Fisher, A2916801) forming a co-culture interface. The next day the medium was substituted by a combination of DMEM and BEGM in a ration 1:1 supplemented with 10 mM CaCl2 (Sigma, Aldrich, C4901). Only when no fibroblasts were used, and the medium was supplemented with 10 ng/ml of IL-10 (Abcam, ab259402), did organoids form branching structures. The organoids were cultured in solution at 37° C. under a humidified atmosphere with 5% CO2 and 95% air. During culture, the medium was refreshed at most every two days.

While the present description has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

1. A process of producing a lung organoid comprising the steps of:
culturing lung cells in an hydrogel and a cell culture medium providing a three-dimensional cell growth medium;
incorporating airway bronchial epithelial cells into the hydrogel at a ratio of at least 2:1 of epithelial cells:lung cells; and
co-culturing the airway bronchial epithelial cells with the lung cells in the hydrogel forming the organoid.

2. The process of claim 1, wherein the lung cells are fibroblast cells.

3. The process of claim 1, wherein the airway bronchial epithelial cells are primary cells or cell lines derived cells.

4. The process of claim 3, wherein the primary cells are patient-derived primary human bronchial airway epithelial cells (pHBE).

5. The process of claim 4, wherein the primary cells are from a patient with an inflammatory condition.

6. The process of claim 1, wherein extracellular matrix (ECM) proteins are incorporated into the hydrogel prior to incorporating the airway epithelial cells.

7. The process of claim 6, wherein the ECM proteins are laminin, collagen IV, entactin, heparin sulfate proteoglycans, or a mixture thereof.

8. The process of claim 1, wherein the cell culture medium comprises a mixture of Minimal Essential Medium (MEM) and Dulbecco's Modified Eagle Medium (DMEM) for LOi or a combination of Bronchial Epithelium Cell Growth Medium (BEGM) and Dulbecco's Modified Eagle Medium (DMEM) for LOp.

9. The process of claim 8, wherein the mixture of MEM:DMEM, or BEGM:DMEM is at a 1:1 ratio.

10. The process of claim 1, wherein the hydrogel comprises alginate, gelatin or a mixture thereof.

11. The process of claim 10, wherein the hydrogel comprises 2% alginate and 5% gelatin.

12. The process of claim 1, wherein the co-culturing of the airway epithelial cells with the lung cells forms spheroids after three days.

13. The process of claim 1, wherein the co-culturing of the airway epithelial cells with the fibroblast cells produces matured organoids in 21 days.

14. The process of claim 1, further comprising incorporating a non-epithelial cell type in the organoid.

15. The process of claim 14, wherein the non-epithelial cell type is monocytes or macrophages.

16. The process of claim 1, wherein the lung organoid is produced using bioprinting.

17. An isolated lung organoid produced by the process of claim 1.

18. (canceled)

19. A kit comprising a surface, an hydrogel coated on said surface, and the organoid of claim 17 grown in the hydrogel.

20. The kit of claim 19, further comprising at least one microfluidic channel casted in the hydrogel.

21. The kit of claim 20, comprising at least one microchannel lined with lung endothelial cells forming a tube and at least one microchannel lined with airway epithelium cells.

22. (canceled)

23. (canceled)

24. (canceled)

25. (canceled)

26. (canceled)

27. (canceled)

28. (canceled)
